(19) United States
(12) Patent Application Publication
(10) Pub. No.: US 2012/0022436 A1
(43) Pub. Date: Jan. 26, 2012

(54) Disposable Environmental Control Chamber for Managing Wounds

(75) Inventors: Richard Bradley, Tarpon Springs, FL (US); Walter F. Leise, Bradenton, FL (US)

(73) Assignee: SARASOTA MEDICAL PRODUCTS, INC., Sarasota, FL (US)

(21) Appl. No.: 13/190,482

(22) Filed: Jul. 26, 2011

Related U.S. Application Data
(60) Provisional application No. 61/400,281, filed on Jul. 26, 2010.
(60) Provisional application No. 61/400,686, filed on Aug. 2, 2010.

(51) Int. Cl. A61M 37/00

(52) U.S. Cl. 604/23

(57) ABSTRACT

The portable environmental control chamber provides gas, as needed, to encourage healing and prevent infection. In one embodiment, the environmental control chamber acts as a hyperbaric chamber that can be affixed over a wound to supply oxygen to the wound bed. In another embodiment, the environmental control chamber acts as a catheter tent that prevents microbes from entering the catheter site. The environmental control chamber comprises a gas impermeable film that forms a tent over a wound or catheter site to which is attached two or more flexible ports that allow gas to be applied to a site. The chamber is attached to the body using an adhesive.

### RELATED APPLICATIONS

This application claims priority to U.S. Provisional Applications 61/400,281 and 61/400,686 filed on Jul. 26, 201 and Aug. 2, 2010, respectively.

### FIELD OF THE INVENTION

The invention relates to a portable environmental control chamber that provides gas, as needed, to encourage healing and prevent infection. In one embodiment, the environmental control chamber acts as a hyperbaric chamber that can be affixed over a wound to supply oxygen to the wound bed. In another embodiment, the environmental control chamber acts as a catheter tent that prevents microbes from entering the catheter site. The environmental control chamber comprises a gas impermeable film that forms a tent over a wound or catheter site to which is attached two or more flexible ports that allow gas to be applied to a site. In the case of the hyperbaric chamber embodiment, oxygen is applied to a wound at a pressure sufficient to enrich the oxygen concentration near the wound bed but low enough to prevent systemic distribution of the oxygen to the patient. In the case of the catheter tent, an inert gas, such as nitrogen, is applied to the catheter connection site at a third port, which is attached to the catheter. This positive pressure inhibits microbe entry.

### BACKGROUND OF THE INVENTION

Some chronic wounds, like leg ulcers, bedsores, and diabetic foot ulcers often resist the healing environment of wound dressings. Although the reason for this is unclear, there is medical evidence that some of these wounds do respond to oxygen treatment. For decades, hyperbaric oxygen treatments have been used as a therapy of last resort when such wounds threaten the life of the patient. Many of these wounds respond and can be subsequently treated by more conventional dressings. The oxygen is believed to jump-start the growth of capillaries in the wound bed that begin the wound healing process. This is called angiogenesis, the absence of which causes a wound to be chronic.

The standard hyperbaric chamber used to provide oxygen to the wound bed is similar to those used to treat underwater divers for the “bends.” They can be found in hospitals and wound care treatment centers throughout the United States. They typically operate at oxygen pressures of seven or more atmospheres. (Normal pressure is one atmosphere.) The problem when healing a wound is that this pressure will drive the oxygen into the bloodstream of the patient. Often these oxygen molecules cause embolisms, stroke and epileptic seizures. They have sometimes even caused patient deaths. Thus, there is a need for topical oxygen administration, which is defined by the U.S. Food and Drug Administration as that being delivered by a medical device at pressures that are insufficient to cause oxygen to be driven into the blood stream. It is non-systemic. It is however from a scientific basis, still hyperbaric because it is at a pressure greater than one atmosphere but significantly less pressure than the pressure required to force the oxygen into the blood vessels.

Hyperbaric chambers operating at only modestly elevated pressure to deliver topical oxygen have been manufactured. One such device, operating at 22 mm Hg pure oxygen (1.03 bar) is shown in B. H. Fischer, “Treatment of Ulcers on the Legs with Hyperbaric Oxygen”, J. OF DERM. SURG., 1:3, October 1975, pp. 55-58, on page 56. However, as indicated in M. C. Y. Heng et al., “A Simplified Hyperbaric Oxygen Technique for Leg Ulcers”, ARCH. DERMATOL., Vol 120, May 1984, pp. 640-645, these chambers are expensive and difficult to sterilize.

Heng and others have proposed a simple hyperbaric oxygen treatment chamber consisting of a polyethylene bag that may be secured to the body or extremity with adhesive tape, or a transparent nylon bag with straps and VELCRO™ closure. S. Olejniczak et al., “Topical Oxygen Promotes Healing of Leg Ulcers”, MEDICAL TIMES, Vol. 104, No. 12, pp. 114-121. Pressure is maintained at between 20 mm Hg and 30 mm Hg; however, there is significant leakage associated with the sealed bag, which requires a relatively high rate of oxygen flow to have effective concentration. As such, this method is useful only in facilities with sufficient oxygen supply or in controlled home environments where a large oxygen tank is permissible.

U.S. Pat. No. 4,875,483 to Alvarez discloses and claims a multi-layered dressing having an external low oxygen-permeability layer and an abutting internal oxygen permeable layer. The relatively low permeability exterior layer is left attached for 3 to 72 hours creating hypoxia and then the layer is removed. The remaining exterior layer is oxygen permeable; however, the layer nevertheless decreases oxygen transport. As such, hyperbaric treatment by another method may be necessary to elevate oxygen levels sufficiently to provide optimal healing.

U.S. Pat. No. 5,029,579 to Trammell discloses and claims a disposable hyperbaric treatment bag that fits over the limb containing the wound. It uses a cuff to secure the chamber to the appendage. This chamber still exposes more of the body than necessary to the oxygen. Further, the functionality and portability is limited because it cannot be localized over the wound itself on flat surfaces of the body, such as the abdomen.

A number of patents have been issued that disclose the use of local generation of oxygen at the wound site to treat wounds in bandage systems using chemical reactions, oxygen saturated solutions, or electrochemical generators (see U.S. Pat. Nos. 5,855,570, 5,578,022, 5,788,682, 5,792,090 and 6,000,403). The devices and methods in these patents however may have insufficient oxygen supply at the wound site due to failure of the chemical reaction.

U.S. Pat. Nos. 7,014,630, 7,263,814, and 7,762,045 to Rosati disclose and claim an apparatus for supplying one or more gases that comprises layers that are sealed on the perimeter to form a reservoir that is gas impermeable on the top layer, but permeable on the bottom layer where it is exposed to the wound. The pressure supply in the bandage is not constant.

While each of the foregoing provides delivery of oxygen to the wound, there are limitations with each, including portability, pressure control (both hyper and hypo) and consistency of oxygen supply. As such, there is a need for an environmental control chamber that is portable, which can be applied more directly to the wound in need of the oxygen with constant oxygen at sufficient pressures for healing without systemic contamination or chemical contamination. It is desirable for such environmental control chamber to also promote hypoxia. It would further be desirable to provide an environmental control chamber that can provide both the benefits of hypoxia and hyperoxia under these conditions by intermittently raising and lowering the oxygen level within the chamber with or without the presence of a hydrocolloid adhesive dressing.

The present invention is portable and provides topical oxygen to a chronic wound at less than two atmospheres where it begins the healing process while avoiding systemic contamination. The oxygen administration in the present invention is controlled with pressure clips and may be continuous or static creating a hyperbaric (or hypoxic) environment, as needed.

In another embodiment, the environmental control chamber acts as a portable mini clean-room and serves to protect the catheter connection and/or wound site from bacteria and environmental contaminants by providing positive pressure.

Other features, advantages, and objects of the invention will become apparent from the specification and figures.

### SUMMARY OF THE INVENTION

The environmental control chamber may function as either a one-piece or a two-piece device. As a one-piece device, it comprises a gas impermeable film (10) and a contiguous adhesive border (30) around the perimeter which is optionally covered by a release liner (40). Attached to the gas film (10) are two or more flexible ports (20) that provide an inlet and an outlet for gas, such as oxygen, and optionally a third port for connecting to a catheter site. The chamber is placed over the wound or catheter connection site to provide a gas rich environment.

As a two-piece device, the environmental control chamber comprises an impermeable film (10), a removal system (60), an adhesive border (30) and an optional release film (40). The removal system (60) may be any system such as VELCRO™ or a female-male flange that allows the top chamber (comprising the top side of the flange (62), the attached film (10) and ports (20)) to be removed while the bottom connector (comprising the body side flange (64) and the adhesive (30)) remain on the patient. The film (10) is attached to the top side of the removal system (60) using an appropriate method such as gluing, molding or welding that provides and maintains the gas tight system. The film (10) should be clear to allow observation of the wound and monitoring of healing. The ports (20) are attached to either the film (10) or the top side of the removal system (60) using an appropriate method such as gluing, molding or welding that provides the gas tight system. The adhesive border is any medical grade adhesive, preferably a hydrocolloid, which is attached to the bottom surface of the film (10) or the body side of the removal system (60) using glue or any appropriate method that maintains the adhesion between the two components. The release film (10) is a liner that is optionally attached to the bottom side of the adhesive to maintain sterility, and adhesive properties until the device is ready for attachment to the patient.

The removal system (60), particularly when it is a female-male flange, acts like TUPPERWARE™. It allows the top side flange (62) to be removed while the body side flange (64) with the adhesive remains on the patient. The chamber top, which comprises the top side (62) of the removal system (60), the film (10) and the ports (20), is removed to allow cleaning of the wound, observation of healing, and changing of a hydrocolloid dressing, if any, while minimizing the loss of barrier properties and providing comfort to the patient. The two-piece device extends the life of the environmental control chamber. The one-piece device is expected to have a life of up to three days, dependent upon the amount of wound exudate produced. The two-piece device is expected to have a life of up to seven days. (The chamber top and any accompanying dressing may be replaced without removing the bottom connector). The environmental control chamber may provide gas to the wound site in either a continuous flow by leaving the gas outlet tube open or it may, by closing the outlet tube, fill the chamber.

The gas film (10) may be any film that is impermeable to gas such as polyurethane, polyvinylidene fluoride, thermoplastic rubber, polyester (Mylar), including multi-layer laminates. The gas impermeable film (10) may be vacuum drawn to provide an expandable pocket for acceptance of the gas. The chamber may be any normal geometric shape consistent with the function of the device and the comfort of the patient. When the removal system (60) is a flange, it may be any flexible medical grade plastic that maintains its shape, such as polyethylene or EVA. In such cases, the gas impermeable film (10) is glued, welded or molded to the top side of the flange.

The environmental control chamber is anchored to the skin surrounding the wound using any medical grade adhesive that can provide a reasonably tight barrier. In the case of the one-piece device, the adhesive (30) is attached directly to the formed gas impermeable film (10). In the case of the two-piece device, the adhesive (30) is attached to the bottom side of the removal system (60) using glue, welding, melding, or other appropriate attachment method. The medical grade adhesive may be a hydrocolloid adhesive that also acts as a gas barrier and provides a physical barrier to microbes and other detrimental environmental assaults.

Hydrocolloids are known to function well as occlusive dressings and also proven to be effective bacterial barriers. Hydrocolloid dressings also provide additional healing properties, absorb wound exudate, and prolong the useful life of the environmental control chamber. As such, in another embodiment, an optional hydrocolloid dressing may be applied directly over the wound in combination with the environmental control chamber. The hydrocolloid may be formulated to have antibacterial properties.

Hydrocolloid adhesive compositions are well known in the art; however, a composition comprised of, as an example, at least one physically cross-linked elastomer selected from the group consisting of styrene-olefin-styrene block copolymer, butyl rubber and ethylene-propylene block copolymers, forming a continuous phase and at least one hydrocolloid dispersed therein, said hydrocolloid being selected from the group consisting of sodium carboxymethyl cellulose, pectin, gelatin, gaur gum, xantham gum, karaya gum, sodium polyacrylate and mixtures thereof, said compositions also containing at least one hydrocarbon tackifier selected from the group consisting of polymers and copolymers of alpha- pinene, beta-pinene, dicylopentadiene and also containing one or more hydrogenated esters of rosin. The adhesive may be made into a more effective antibacterial by incorporating antimicrobial substances such as silver salts and silver ceramic compounds.

The environmental control chamber may be made more comfortable by adding a layer of foam. The adhesive may be applied to a closed cell or reticulated foam that allows a physical cushioning of the device. The foam may also contain antibacterial agents, such as ionic silver to provide even more protection for the healing wound.

The environmental control chamber of the present invention simplifies and miniaturizes the application of a gas to a wound site. Now, instead of confining the patient to a chamber, the environmental control chamber is applied directly over the wound using any medical grade adhesive. In so doing, the environmental control chamber is applying positive pressure. In another embodiment, the environmental control chamber may be used as a catheter tent. In this embodiment, as with the one-piece hyperbaric chamber embodiment, the catheter tent comprises a gas impermeable film (10) having three ports (20) (input, output, catheter connector) with an adhesive border (30) around the perimeter which is optionally covered by a release liner (40). In the case of a two-piece catheter tent, it comprises an impermeable film (10), a removal system (60), three ports (20) (input, output, catheter connector), an adhesive border (30) attached to the bottom side of the removal system (60) and an optional release liner (40). The input and output ports (20a and 20b) in this embodiment function as they did in the hyperbaric chamber embodiment. The third port (20c) provides a connection for the catheter and in some instances provides a delivery route to the catheter. As with the hyperbaric chamber embodiment, application of a gas may inflate the chamber top, thus providing positive pressure around the catheter connection and the wound site. The positive pressure generated within the tent provides a barrier to bacterial invasion in the catheter and diminishes the chances of hospital acquired infections, which are a problem in all hospitals.

Hospital acquired infections are particularly prevalent with indwelling catheters (feeding tubes, etc.) required for patient care. It is not uncommon to coat the lumen of catheters with antibacterial solutions, such as silver, to prevent intrinsic infection, which results from bacteria driving down the lumen of a catheter or tube. However, the extrinsic infections that occur when bacteria grow on the outside surface of the catheter and its biofilm have not been adequately addressed. Further, as the patient moves, or is moved, slippage of the catheter can inoculate the patient with contaminants. The catheter embodiment of the present invention addresses the issue by locking the catheter in place with a barbed fitting and simultaneously providing positive pressure containment around the insertion site and connection section. The flexible design provides patient comfort and ease of use by the healthcare giver.

In general, the environmental control chamber of the present invention provides gas to a wound site to aid healing and curtail infections.

### BRIEF DESCRIPTION OF THE DRAWINGS

Presently preferred embodiments of the invention are described below in conjunction with the appended drawing figures, wherein like reference numerals refer to like elements in the various FIGS., and wherein:

FIG. 1A is a perspective view of the top of the one-piece environmental control chamber;

FIG. 1AB is a perspective view of the side of the one-piece environmental control chamber;

FIG. 2A is an exploded view of the two-piece environmental control chamber;

FIG. 2B is a perspective view of the top of the two-piece environmental control chamber;

FIG. 3 is a perspective view of the top of the two-piece environmental control chamber being separated; and

FIG. 4 is a perspective view of the two-piece catheter tent.

### DETAILED DESCRIPTION OF THE INVENTION

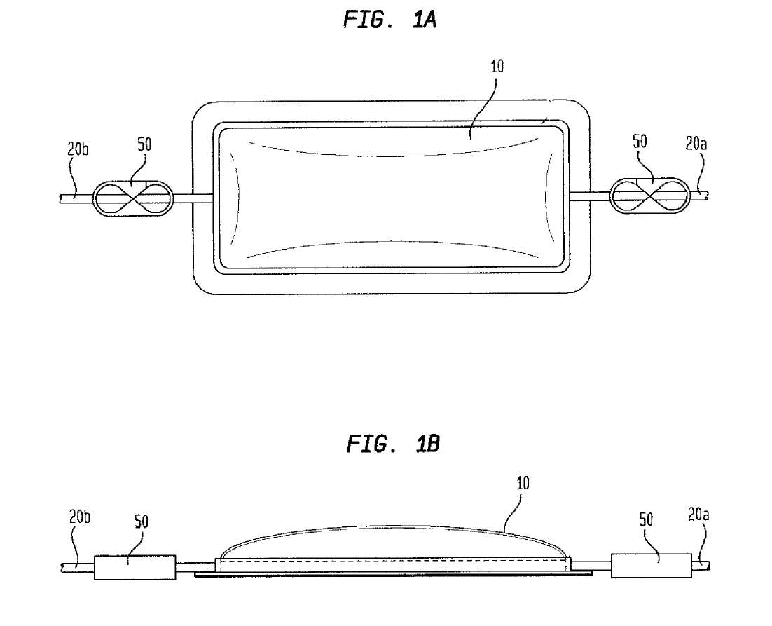

The present invention provides a portable environmental control chamber which provides gas, as needed, to encourage healing and prevent infection at a wound site. The environmental control chamber, acting as a one-piece device as show in FIG. 1A, comprises a clear gas impermeable continuous film (10) having two ports (20) that provide an inlet (20a) and an outlet (20b) for gas and optionally a third port (20c) for connecting to a catheter; and a contiguous adhesive border (30) applied to the inner perimeter of the film (10), wherein the adhesive border (30) is optionally covered by a release liner (40). The chamber is placed over the wound to provide a gas rich environment. The gas is applied in either a continuous or non-continuous fashion with a pressure clip (50). The gas may be any gas that meets the intended purposes. In the hyperbaric chamber, the gas is preferably oxygen. In the catheter tent, the gas is preferably an inert gas. Most preferably, the gas is nitrogen.

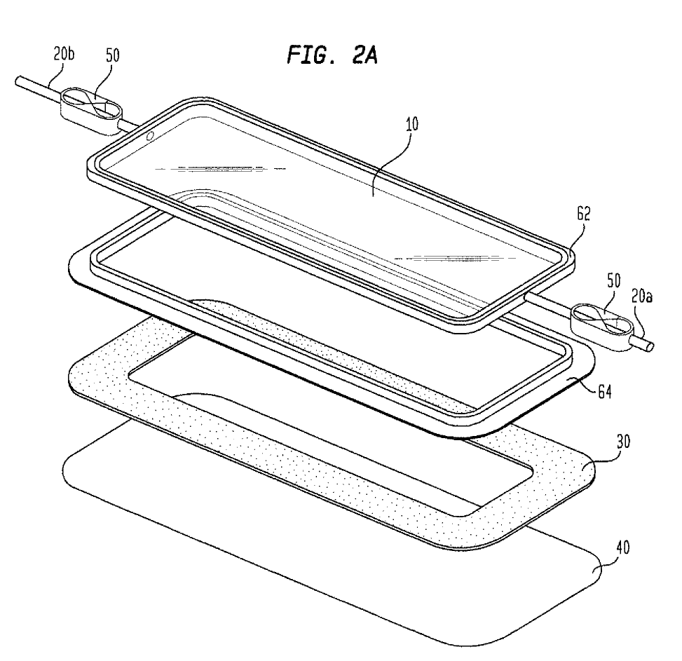

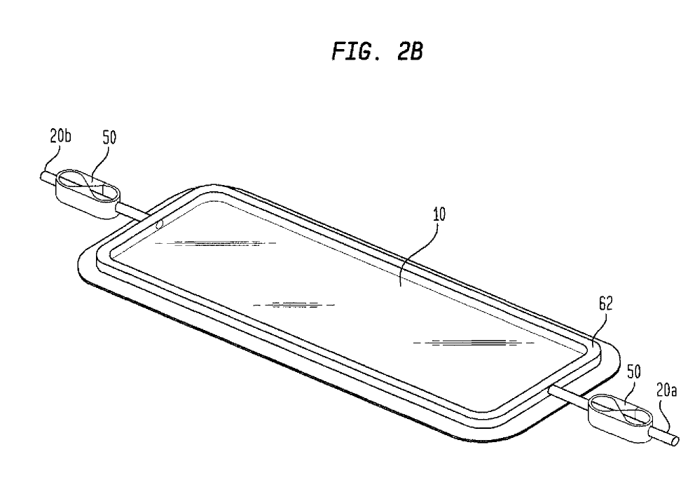

In another embodiment of the present invention, the environmental control chamber is a two piece device as shown in FIGS. 2A, 2B, 3 and 4, comprising a clear gas impermeable continuous film (10) and having a gas inlet port (20a) and a gas outlet port (20b) and optionally a third port (20c) for connecting to a catheter, wherein said clear gas impermeable film (10) is attached to a removal system (60) having a top side (62) and a body side (64) to which is attached an adhesive (30), and an optional release liner (40).

The gas film (10) may be any film that is clear and impermeable to gas selected from the group consisting of polyurethane, polyvinylidene fluoride, thermoplastic rubber, polyester, and multi-layer laminates. The gas impermeable film (10) may be vacuum drawn to provide an expandable pocket for acceptance of the gas as shown in FIG. 1B. The film (10) may be taught as shown in FIGS. 2A, 2B, 3 and 4 or it may be an expandable pocket that provides a cavity for the gas such as shown FIGS. 1A and 1B. Preferably, the film (10) is flexible to provide comfort to the patient. One skilled in the art will appreciate that the film (10) may be any width that allows it to remain clear, expandable, flexible and impermeable to gas.

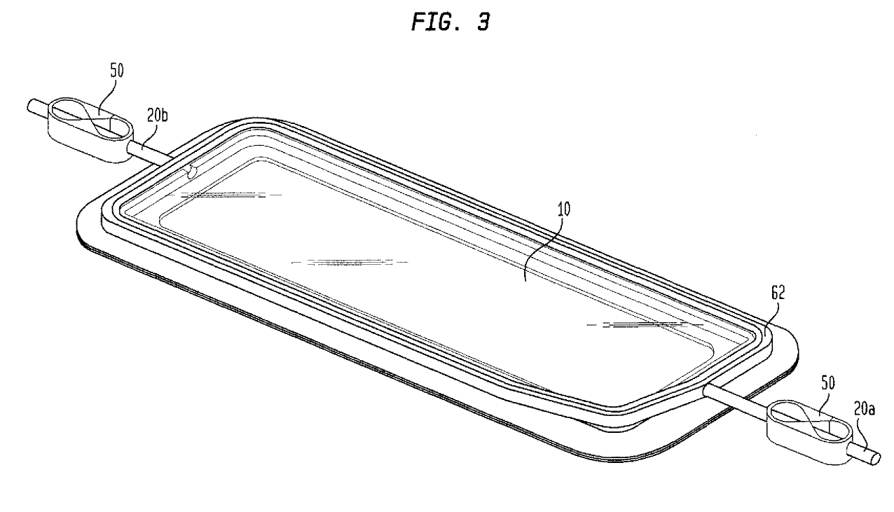

The removal system (60) may be any system that allows the top side (62) and body side (64) to be separated, such as VELCRO™ or a male-female flange. As shown in FIG. 3, the removal system (60) is preferably a flange system having a top side (62) and a body side (64) that allows the top side (62) flange to be removed while the body side (64) flange with the adhesive (30) remains on the patient. The chamber top, which comprises the top side flange (62), the film (10) and the ports (20), is removed to allow cleaning of the wound and changing of a separate hydrocolloid dressing, if any, while minimizing the loss of barrier properties and providing comfort to the patient. The two-piece environmental control chamber may be used continuously up to seven days. The one-piece device may be used continuously for up to three days. The male-female flange may be any flexible plastic that maintains its shape, such as polyethylene or EVA.

The clear gas impermeable film (10) is attached to the removal system (60) on the top side (62), either the interior or exterior. One skilled in the art will appreciate that the attachment method is dependent upon the material used for the removal system (60) but may be any method that provides a complete seal between the film (10) and the top side (62) of the removal system (60). In to preferred embodiment, the gas impermeable film (10) is welded or molded to the top side (62) of the male-flange.

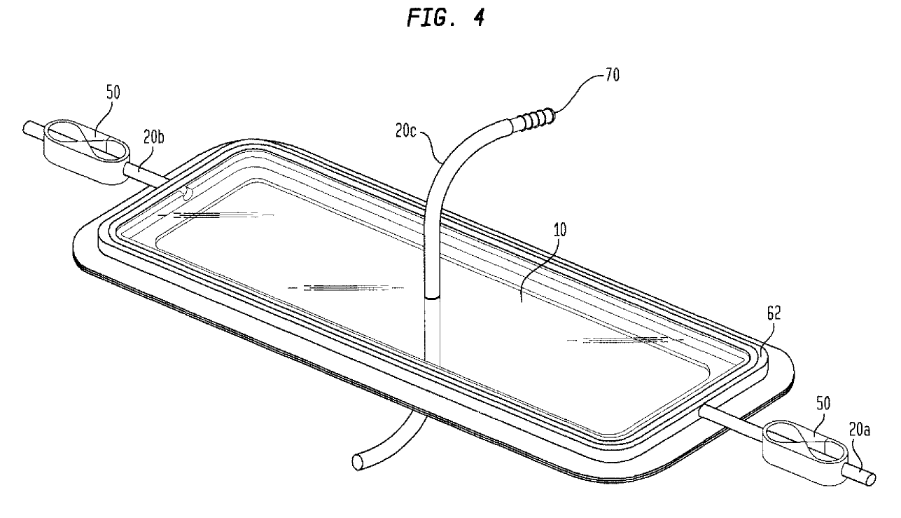

The inlet port (20a) and outlet port (20b) are any medical grade material that allows the free flow of gas into the cavity of the chamber. Preferably, the ports (20) are flexible, which aids in the portability of the environmental control chamber and comfort to the patient. As shown in FIG. 4, the connection port (20c) is made in a width to allow connection to a catheter. In this embodiment, the connector port (20c) also has a barbed fitting (70) for secure fastening to the catheter. The ports (20) may be of equal or different widths depending on the purpose of the connection. The length of the ports (20) may vary depending on the distance from the connection site of the gas supply and/or catheter. In the one-piece embodiment, the ports (20) are attached to the gas impermeable continuous film (10) by molding, welding, or some other method that provides a complete seal between the film (10) and the ports (20). In the two-piece embodiment, the ports (20) may be attached to the gas impermeable continuous film (10) or to the removal system (60). When attached to the removal system (60), the ports (20) are attached to the top side (62) of the removal system using molding or welding, or some other method that provides a complete seal between the port (20) and the top side (62) of the removal system (60).

The environmental control chamber is attached to the patient using an adhesive (30). In the one-piece embodiment, the adhesive (30) is applied to the inner perimeter of the clear gas impermeable film (10) using glue, welding or any other appropriate method that maintains the adhesion between the two components. In the two-piece device, the adhesive (30) is applied to the bottom surface of the body side (64) of the removal system (60) using glue or any appropriate method that maintains the adhesion between the two components. Preferably, the adhesive (30) is applied to the bottom surface of the flange (64). It will be understood that any suitable non-toxic adhesive for use in medical purposes can be used for this purpose. Preferably, the adhesive (30) is pressure sensitive. Most preferably, the adhesive (30) is a hydrocolloid, which also acts as a gas barrier (reinforcing the impermeability of the chamber) and provides a physical barrier to microbes and other detrimental environmental assaults.

Hydrocolloid adhesive compositions are well known in the art; however, as an example, a composition be comprised of at least one physically cross-linked elastomer selected from the group consisting of styrene-olefin-styrene block copolymer, butyl rubber and ethylene-propylene block copolymers, forming a continuous phase and at least one hydrocolloid dispersed therein, said hydrocolloid being selected from the group consisting of sodium carboxymethyl cellulose, pectin, gelatin, gaur gum, xantham gum, karaya gum, sodium polyacrylate and mixtures thereof, said compositions also containing at least one hydrocarbon tackifier selected from the group consisting of polymers and copolymers of alpha- pinene, beta-pinene, dicylopentadiene and also containing one or more hydrogenated esters of rosin. Such compositions may be made into more effective antibacterial compositions through the incorporation of antimicrobial substances as for example silver salts and silver ceramic compounds. The adhesive may first be applied to a closed cell, or reticulated foam, which allows a physical cushioning of the chamber. The adhesive may be applied to a closed cell or reticulated foam that allows a physical cushioning of the device. Such foam may contain antibacterial agents, as for example ionic silver, to provide additional protection for the healing wound.

Hydrocolloids are known to function well as occlusive dressings and also proven to be effective bacterial barriers. As such, in another embodiment, an optional hydrocolloid dressing may be applied directly over the wound in combination with the environmental control chamber. The hydrocolloid may be formulated to have antibacterial properties. The optional hydrocolloid dressing provides additional healing properties, absorbs wound exudate, and prolongs the life of the environmental control chamber.

The adhesive border (30) and any optional dressing is optionally covered by a release liner (40), which maintains sterility and adhesive properties until the device is ready for attachment to the patient. In the case of the one-piece device it is attached to bottom side of the adhesive (30) which is attached directly to the film (10). In the case of the two-piece, it is attached to the bottom of the adhesive (30) that is attached to the bottom side of the body side (62) of the removal system.

The male-female flange removal system (60) acts like a TUPPERWARE™, which allows the top side flange (62) to be removed while the body side flange (64), to which an adhesive (30) is attached, remains on the patient. The chamber top, which comprises the top side flange (62), the film (10) and the ports (20), is removed to allow cleaning of the wound and changing of a separate hydrocolloid dressing while minimizing the loss of barrier properties and providing comfort to the patient. The two-piece device may be used continuously up to seven days, whereas the one-piece device may be used continuously for up to three days. The environmental control chamber may provide gas to the wound site in either a continuous flow by leaving the gas outlet port (20b) open or it may, by closing the gas outlet port (20b), fill the chamber for a constant application of pressure. A hypoxic condition can be created by emptying the chamber and stopping the oxygen supply.

The chamber is placed over the wound to provide an oxygen rich environment. In one embodiment, oxygen is delivered through the inlet port (20a) until the chamber is fully pressurized. In this case, the outlet port (20b) is closed with one or more pressure control clips (50). One skilled in the art will appreciate that while this is described as a two port system, a single port system could also be used. In other words, a single port (20) could serve the function for both input and outflow of the oxygen. In such case, the oxygen would be released upon opening of the pressure clip (50) or removal of the chamber top or the device in its entirety from the body. In another embodiment, the oxygen can be applied in a continuous fashion. In such case, the oxygen is delivered to the inlet port (20a) and discharged through the outlet port (20b) continuously. During the continuous application of oxygen, the pressure clips (50) on each of the inlet port (20a) and outlet port (20b) will be open. The source of the oxygen can be from any external supply, such as a “house” gas line as is found in a hospital and some nursing home facilities. It can also be obtained from oxygen tank, an oxygen generator, an oxygen concentrator, or oxygen produced from perflorocarbons or other in situ applications.

The chamber may also be used over a catheter, intravenous line or wound insertion site to prevent infection at the site. While in the embodiment described above, the environmental control chamber acts as a hyperbaric chamber, one skilled in the art will appreciate that the catheter tent may utilize any inert gas that provides a positive pressure at the site. Preferably, the gas is nitrogen.

As with the hyperbaric chamber, the catheter tent comprises a clear gas impermeable film (10) having a contiguous adhesive border (30) around the perimeter which is optionally covered by a release liner (40) and three ports (20) (a gas input port (20a); a gas output port (20b); and a connection port (20c)) attached to the film (10) in the case of a one-piece device or the removal system (60) in the case of a two-piece device. The connection port (20c) is attached to the gas impermeable continuous film (10) by molding, welding, or some other method that provides a complete seal between the film (10) and the port (20c). The connection port (20c) may stop at the film (10) or extend beyond the film and provide access to the catheter. In all cases, the input and output ports (20) have a supply and pressure control clip (50) that fits over the ports and control the flow of gas into and out of the chamber cavity. In the case where the distal end of the connection port (20c) extends beyond the continuous film, it may also have a clip that is attached at the distal end, but preferably has a valve that seals the distal end of the port closed when not in use. Attached to the proximal end of the connector port (20c) is a barbed fitting (70) for secure fastening to the catheter. Upon application of a gas through the inlet port (20a), the catheter tent becomes a mini-clean room, prohibiting the entry of microbes along the exterior of the port. The lumen of the port may be coated with antibacterial solutions, such as silver, to prevent intrinsic infection, which results from bacteria driving down the lumen of a catheter or tube.

The catheter tent top, which comprises the top side flange (62), the film (10) and the ports (20), may be removed to allow the catheter to be changed, while minimizing the loss of barrier properties and providing comfort to the patient. The connection port (20c) may also enable catheter access without breaking the positive pressure seal. This allows all medical procedures to be done without breaking the positive pressure seal.

The environmental control chamber may be any geometric shape, including but not limited to a square, rectangle, or circle, consistent with the function of the device, the wound to be treated and the comfort of the patient.

While the foregoing discloses the preferred embodiments of the invention, it will be understood by those skilled in the art that many of the details may be varied without departing form the spirit and scope of the invention.

We claim:

1. A portable environmental control chamber comprising:
(a) a gas impermeable continuous film;
(b) two or more flexible plastic ports that provide an inlet and an outlet for gas flow, having a pressure clip attached thereto, wherein said ports are attached to said continuous film; and
(c) an adhesive border applied to the inner perimeter of the film, wherein the adhesive border is optionally covered by a release liner.

2. The portable environmental control chamber of claim 1, wherein the film is selected from the group consisting of polyurethane; polyvinylidene fluoride; thermoplastic rubber; polyester; and multi-layer laminates.

3. The portable environmental control chamber of claim 1, wherein the film is vacuum drawn to provide an expandable pocket for acceptance of a gas.

4. The portable environmental chamber of claim 1, wherein the adhesive is a hydrocolloid adhesive.

5. The portable environmental chamber of claim 1, wherein the adhesive is first applied to a foam.

6. The portable environmental chamber of claim 1, wherein the gas applied is oxygen or nitrogen.

7. A portable environmental control chamber comprising:
(1) a clear gas impermeable continuous film;
(b) a removal system having a top side and a body side, where the film is attached to the top side of the removal system;
(c) two or more flexible ports, wherein the ports are attached either of the top side of the removal system or to the continuous film;
(d) an adhesive border, wherein the adhesive is attached to the bottom of the body side of the removal system; and
(e) an optional release liner, wherein the optional releaser line is attached to the adhesive.

8. The portable environmental control chamber of claim 7, wherein the film is selected from the group consisting of polyurethane; polyvinylidene fluoride; thermoplastic rubber; polyester; and multi-layer laminates.

9. The portable environmental control chamber of claim 7, wherein the film is vacuum drawn to provide an expandable pocket for acceptance of a gas.

10. The portable environmental chamber of claim 7, wherein the adhesive is a hydrocolloid adhesive.

11. The portable environmental chamber of claim 7, wherein the adhesive is first applied to a foam.

12. The portable environmental chamber of claim 7, wherein the gas applied is oxygen or nitrogen.

13. The portable environmental chamber of claim 7, further comprising an independent hydrocolloid dressing.

14. The portable environmental chamber of claim 13, wherein said hydrocolloid dressing contains antimicrobial agents.

15. The portable environmental chamber of claim 7, wherein one the ports has attached thereto a barbed fitting.

16. A method of applying gas to a wound comprising the steps of:
a. applying an environmental control chamber over a wound of a patient, wherein said environmental control chamber comprises:
(i) a gas impermeable continuous film;
(ii) one or more flexible ports that provide an inlet and an outlet for gas flow, having a pressure clip attached thereto,
(iii) an optional removal system having a top side and a body side, wherein said ports are attached to the film or the top side of the removal system; and
(iv) an adhesive border applied to the inner perimeter of the film or the bottom of the body of the removal system, wherein the adhesive border is optionally covered by a release liner; and
b. delivering gas to the environmental control chamber wound.

17. The method of applying gas to a wound of claim 16 wherein the gas applied is oxygen or nitrogen.

18. The method of applying gas to a wound of claim 16, wherein the gas applied is applied continuously.

19. The method of applying gas to a wound of claim 16, wherein the gas applied is statically.

20. The method of applying gas to a wound of claim 16, wherein the portable environmental chamber of further comprises a foam attached to the adhesive.
